(19) United States
(12) Patent Application Publication
(10) Pub. No.: US 2023/0272317 A1
(43) Pub. Date: Aug. 31, 2023

(54) PHOTO REACTOR FOR TETRAHYDROCANNABINOL (THC) TESTING

(71) Applicant: Purdue Research Foundation, West Lafayette, IN (US)

(72) Inventors: Charles Steele, Hobart, IN (US); Reynaldo Barreto, New Carlisle, IN (US)

(73) Assignee: Purdue Research Foundation, West Lafayette, IN (US)

(21) Appl. No.: 18/114,675

(22) Filed: Feb. 27, 2023

Related U.S. Application Data
(60) Provisional application No. 63/315,013, filed on Feb. 28, 2022.

(51) Int. Cl. C12M 1/00; B01J 35/00; B01J 19/12

(52) U.S. Cl. CPC C12M 21/02; B01J 35/004; B01J 19/127

(57) ABSTRACT

The photoreactor system includes a chamber, a lid, a catalyst coating, and an oxygen supply port. The photoreactor system is configured to process a sample by breaking down organic molecules, such as Tetrahydrocannabinol (THC). The catalyst coating is coupled to an interior surface of the chamber. The photoreactor system includes a mixing blade to agitate the sample. The chamber also includes a baffle substantially covered with the catalyst coating to enhance the turbulent flow of the sample and provide more catalyst coated surface area within the chamber.

### CROSS REFERENCE TO RELATED APPLICATIONS

The present application is a U.S. non-provisional application which claims the benefit of U.S. provisional application Ser. No. 63/315,013, filed Feb. 28, 2022, the content of which is incorporated by reference herein in its entirety.

### FIELD

The disclosure generally relates to photoreactors and, more particularly, to photoreactors capable of breaking down chromaphore.

### INTRODUCTION

This section provides background information related to the present disclosure which is not necessarily prior art.

With the proliferation of legal Tetrahydrocannabinol (CBD), Hemp, and Marijuana products the United States, the growth of cottage and full-scale industrial manufacturing of products featuring these ingredients has drastically increased. Each of these industry segments has specific testing needs. Of particular concern are CBD, which need to be THC free, and Hemp products, which must be below 0.3% THC. This requirement remains even in areas where recreational marijuana is legal.

Unfortunately, many CBD products are not THC free. Various studies have shown that as many as 50%-70% of CBD products on the market contain measurable amounts of THC. This represents a risk, not only to the manufacturer for compliance and false advertising, but also to the consumer. THC can remain in the blood of the consumer for up to six weeks after it has been consumed. In states like Indiana, THC levels above 5 ng/ml in the blood can result in a DWI conviction. Such a conviction can carry major consequences including jail time and permanent exclusion from professions like medicine and law enforcement. Even if a consumer is not arrested, undesirable consequences remain. For instance, truck drivers may lose their CDL license and students may lose their scholarships after testing positive for THC. In a specific example, middle school children have been hospitalized after eating similar products containing THC.

Manufacturers of CBD products could use commercially available test kits from NIK® to test for and quantify THC. Similarly, the QuanTHC test kit commercially available from CBF FORENSICS LLC. However, these tests are designed for law enforcement. Small commercial users may not have the economical means to dispose of the chemicals used in these tests. The need for the disposal of the test chemicals was identified as a barrier to use of QuanTHC and other available tests for the cottage manufacturers.

In order for such THC tests to useful for boutique and cottage manufacturers, the ability to process the chemicals used in the tests for disposal is necessary. The most common of these chemicals are based on the Duquenois-Levine test which contains some or all of the following: Acetaldehyde (C2H4O), Chloroform (CH2Cl3), Ethanol (C2H5OH), Hydrochloric Acid (HCl), Vanillin (C8H8O3), Δ-9 THC (C21H30O2), and the Duquenois-Levine chromophore (C31H38O3).

To process these chemicals, boutique and cottage manufacturers may perform a photocatalysis reaction. Incident photons of a photocatalytic process may eject valence electrons from titanium dioxide (TiO₂). It is known to use high energy photons in the UVC range because TiO2 has a work function of 4.4 eV, meaning that the UV light needed to eject an electron is in the range of 280 nm±40 nm. It is also known that by introducing oxygen (O₂) into a photocatalysis reaction, a reduction in the work function of the TiO2 may occur. This reduction in work function may allow the use of lower energy photons (400 nm±40 nm) in the UVA range. The ejected electron can then be picked up by an O₂ molecule to provide an O₂₋ and facilitate the formation of a hydroxyl free radicle (HO·). The generated ions can then initiate redox reactions to break harmful organic molecules down into substances that can be released into the environment.

HO· is an extremely short-lived species that has a reported half-life of 1 ns and diffusion limited rate constants on the order of 10⁻⁹ s making the reaction most probable near the surface of the TiO₂. However, known photoreactors include the use of O₂ with UVA which favors the production of superoxide O2−·, which is longer lived and allows for enhanced dispersion through a volume of water.

However, known photoreactors that even utilize O₂, as described above, still have inefficiencies and are often too costly for boutique and cottage manufacturers to procure and operate. Accordingly, there is a continuing need for a photoreactor system that may be more economically utilized while enhancing the efficiency and effectiveness of breaking down the chemical by-products of THC testing kits.

### SUMMARY

In concordance with the instant disclosure, a photoreactor system that effectively breaks down the by-products of THC testing kits, has been surprisingly discovered. Desirably, the photoreactor system may be manufactured and operated more economically than known photoreactors.

The photoreactor system includes a chamber, a lid, a catalyst coating, and an oxygen supply port. The photoreactor system may be configured to break down Tetrahydrocannabinol (THC) from a sample. The chamber may include a sidewall and a bottom wall. The chamber may be configured to accept the sample. The lid may be coupled to the sidewall of the chamber. The lid may be configured to be selectively disposed in at least one of an open position and a closed position. The catalyst coating may include TiO₂. The catalyst coating may be coupled to an inner surface of the sidewall and/or an inner surface of the bottom wall. The oxygen supply port may be coupled to the chamber.

In certain circumstances, the photoreactor system configured to process the sample may include a kit. The kit may include a chamber, a lid, a catalyst coating, and an oxygen supply port. The chamber may include a sidewall and a bottom wall. The chamber may be configured to accept the sample. The lid may be configured to be coupled to the sidewall of the chamber. The lid may be configured to be selectively disposed in an open position and/or a closed position. The catalyst coating including TiO₂ that may be configured to be disposed on an inner surface of the sidewall. The oxygen supply port may be configured to be coupled to the chamber.

Various ways of using the photoreactor system are provided. For instance, a method may include a step of providing the photoreactor system. The method may include a step of providing a sample to be processed. Next, a predetermined volume of water may be inserted into the chamber. Then, oxygen may be injected into the chamber. The method may include a step of disposing the sample into the chamber of the photoreactor system. Afterwards, ultraviolet light may be applied to the sample within the chamber.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a top perspective view of a photoreactor system, according to one embodiment of the present disclosure;

FIG. 2 is a left-side elevational view of the photoreactor system, further depicting a drain valve and an oxygen supply port, according to one embodiment of the present disclosure;

FIG. 3 depicts a top perspective view of a spectrophotometer detector, according to one embodiment of the present disclosure;

FIG. 4 is a left-side elevational view of the spectrophotometer detector, as shown in FIG. 3, according to one embodiment of the present disclosure;

FIG. 5 is a line chart depicting an example GutReise E10 Screw 3V or 4.5V target emission spectra in relation to the spectrophotometer detector, according to one embodiment of the present disclosure;

FIG. 6 is a top plan view of the lid, further depicting a substantially transparent lid that is configured to permit sunlight to pass therethrough, according to one embodiment of the present disclosure;

FIG. 7 is a top plan view of an alternative embodiment of the lid, further depicting a reflective coating and a plurality of ultraviolet lights disposed on an interior surface of the lid, according to one embodiment of the present disclosure; and

FIG. 8 is a flowchart depicting a method for using the photoreactor system, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9,1-8,1-3,1-2,2-10,2-8,2-3,3-10,3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the FIG. is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

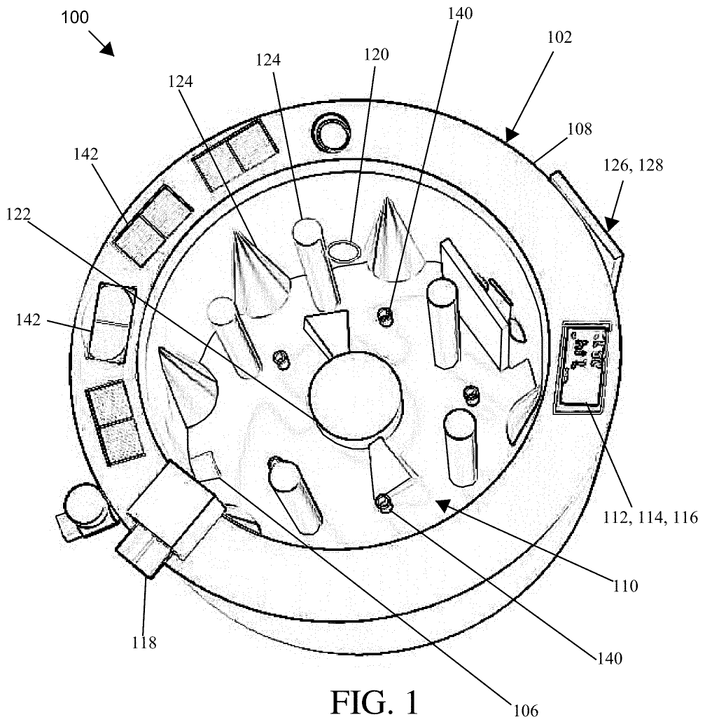

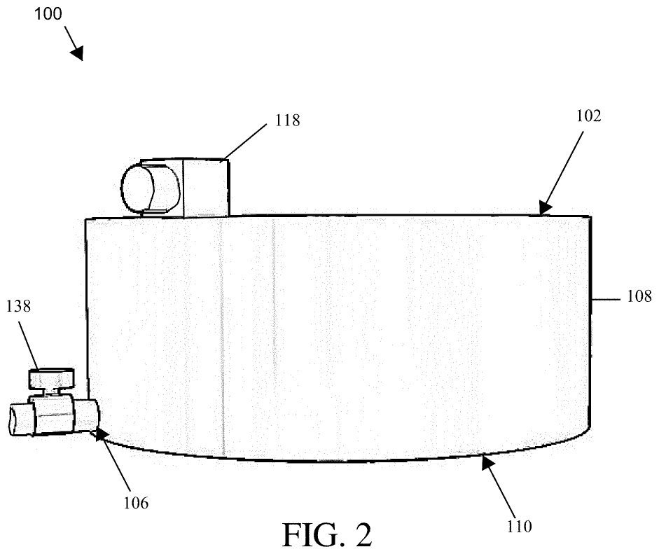

As shown in FIGS. 1-2, the photoreactor system 100 includes a chamber 102, a lid 104, a catalyst coating, and an oxygen supply port 106. The photoreactor system 100 may be configured to break down organic materials, such as Tetrahydrocannabinol (THC), from a sample. The chamber 102 may include a sidewall 108 and a bottom wall 110. The chamber 102 may be configured to accept the sample. The lid 104 may be coupled to the sidewall 108 of the chamber 102. The lid 104 may be configured to be selectively disposed in at least one of an open position and a closed position. The catalyst coating may include titanium dioxide (TiO₂) and/or Barium. The catalyst coating may be coupled to an inner surface of the sidewall 108 and/or an inner surface of the bottom wall 110. The oxygen supply port 106 may be coupled to the chamber 102. In certain circumstances, a solution may be disposed within the chamber 102, such as water. In a specific example, the chamber 102 may be filled with less than around 2.5 inches of the solution. In a non-limiting example, the photoreactor system 100 may be configured as a benchtop photoreactor. For instance, the photoreactor system 100 may be less than around three feet in height and diameter. The photoreactor system 100 may be configured to be utilized by boutique and cottage cannabis product manufacturers as well as small scale production laboratories, quality assurance laboratories, and educational chemistry laboratories.

With reference to FIGS. 1-2, the chamber 102 may have certain functionalities that may be performed and visualized by various components. For example, the chamber 102 may have a screen 112, 114, 116 that may display a variety of information. More specifically, the screen 112, 114, 116 may include a pH meter 112 that is configured to measure a pH value of the sample. The screen 112, 114, 116 may further include a thermometer 114 that is configured to measure a temperature of the sample. The screen 112, 114, 116 may include a timer 116 that advantageously permits a user to have enhanced certainty the reaction is completed where the reaction occurs for a predetermined length of time that is necessary to process the sample. The screen 112, 114, 116 may include a single display providing the results of each of the pH meter 112, the thermometer 114, and/or the timer 116. The chamber 102 may include a hydrogen peroxide inlet. The hydrogen peroxide inlet 118 may be configured to direct added hydrogen peroxide through the hydrogen peroxide inlet 118 disposed on the interior surface of the sidewall 108 of the chamber 102. The hydrogen peroxide inlet 118 may be disposed a height along the interior surface of the sidewall 108 of the chamber 102 that is configured to militate against a backflow of materials exiting the chamber 102 through the port. The chamber 102 may further include a drain valve 120. In a specific example, the drain valve 120 may include a filter that is configured to remove solids and/or particulates from the sample.

With reference to FIG. 1, the chamber 102 may include ways to agitate the sample. For instance, the chamber 102 may include a mixing blade 122. The mixing blade 122 may be a configured to gently swirl water during the reaction. Additionally, the chamber 102 may include a baffle 124 on the inner surface of the sidewall 108 and/or the inner surface of the bottom wall 110. The baffle 124 may also be coated with the catalyst coating including TiO₂. The baffle 124 may be selectively removeable from the chamber 102. For instance, the baffle 124 may include a pin that is configured to be accepted via a friction fit design in an aperture located in the bottom wall 110 of the chamber 102. In a specific example, the baffle 124 may be substantially cylindrically shaped and/or substantially conically shaped. Advantageously, the mixing blade 122 and the baffle 124 may enhance the turbulent flow and the surface area of the catalyst coating including TiO₂, thereby further enhancing the processing of the sample.

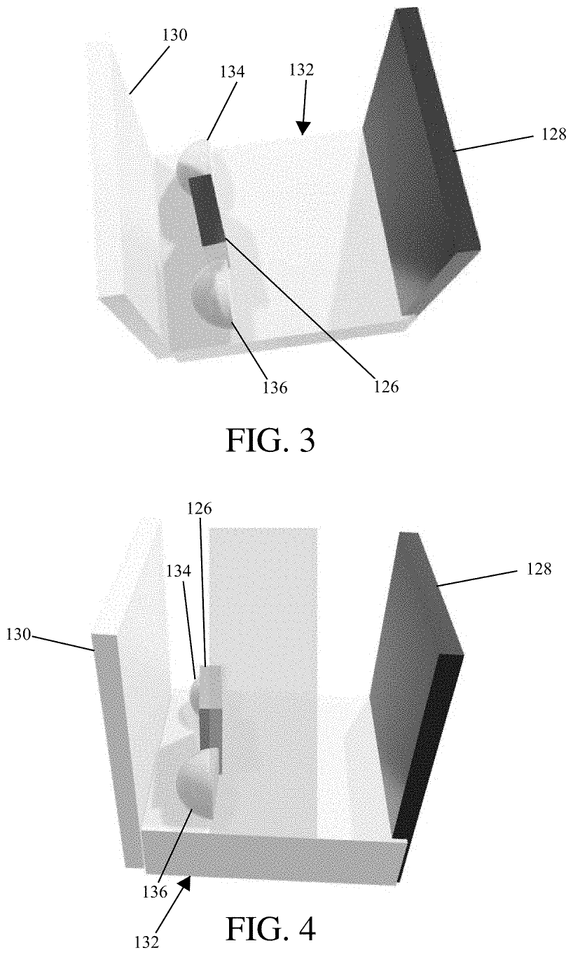

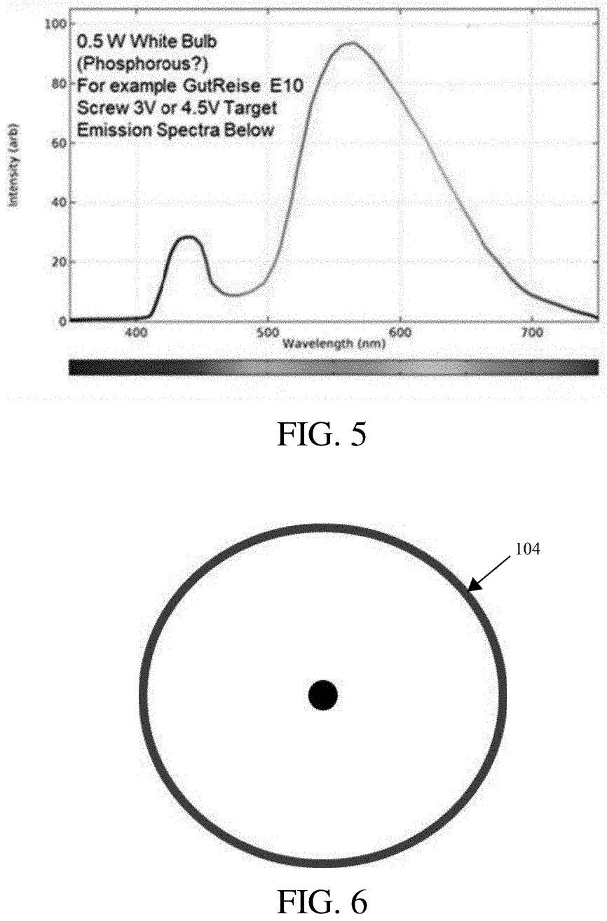

As shown in FIGS. 1 and 3-5, the photoreactor system 100 may include a way to analyze the sample while the sample is being processed. For instance, the photoreactor system 100 may include a spectrophotometric detector 126, 128. The spectrophotometric detector 126, 128 may be an instrument that is configured to measure a number of photons (the intensity of light) absorbed after it passes through the sample solution. With the spectrophotometer detector 126, 128, the amount of a known chemical substance/concentration may also be determined by measuring the intensity of light detected. The spectrophotometer detector 126, 128 may include a display that is configured to provide data to a user. In a particular embodiment, the spectrophotometric detector 126, 128 may include a view port 126 disposed on the chamber 102 and/or the lid 104 of the photoreactor system 100. In a specific example, the view port 126 may include a device view port and a device holder 128. The device holder 128 may be configured to support a cellphone, tablet, or other computer capable of detection. The view port 126 may include a transparent lens. The view port 126 may be configured to allow a camera of a mobile device and/or computer to observe the solution of the chamber 102. Provided as non-limiting examples, the mobile device holder 128 may include a friction fit coupling, a threaded coupling, a mechanical fastener, and/or a hook-and-loop coupling to couple the mobile device to the chamber 102. Advantageously, the mobile device holder 128 may be configured to repeatably couple a mobile device to the chamber 102 in a substantially similar position and angle in comparison to the chamber 102. In a specific example, the mobile device and/or computer may include an application configured to provide spectrophotometric analysis. The spectrophotometric detector 126, 128 may include a reflective plate 130 disposed within the chamber 102. The reflective plate 130 may include an L-shaped flange 132 depending from the interior surface of the sidewall 108 of the chamber 102. The reflective plate 130 may be coated with TiO₂ and/or Barium. The reflective plate 130 may be substantially parallel with the view port 126. The view port 126 may be a transparent lens and further include a light source 134, 136. The reflective plate 130 may be disposed inside of the chamber 102, facing the light source 134, 136 and the view port 126. The transparent lens may enhance the view of the reflective plate 130. In a specific example, the light source 134, 136 may provide white light. The light source 134, 136 may be configured to emit light with a range up to around 570 nm to 590 nm. In a specific example, the light source 134, 136 may include a pair of white lights disposed on terminal sides of the transparent lens with each white light directed toward the reflective plate. In other words, the light source 134, 136 may include a first powered light 134 and a second powered light 136, wherein the reflective plate 130 is disposed in between the first powered light 134 and the second powered light 136. Desirably, the positioning of the pair of light sources 134, 136 may provide a more evenly distributed illumination of light on the reflective plate 130. A skilled artisan may select other suitable ways of providing a spectrophotometer detector 126, 128 on the photoreactor system 100, within the scope of the present disclosure.

In certain circumstances, the oxygen supply port 106 may include various features. For instance, as shown in FIG. 1, the oxygen supply port 106 may be configured to be coupled to an air pump 138. The air pump 138 may be used to inject ambient air into the chamber 102. In another specific example, the oxygen supply port 106 may be configured to be coupled to an oxygen tank. The oxygen tank may be used to inject pure oxygen into the chamber 102. In a particular embodiment, the oxygen supply port 106 may be coupled to exhaust holes in the bottom wall 110 of the chamber 102. The ambient air and/or pure oxygen disposed through the oxygen supply port 106 may be configured to be emitted through the exhaust holes 140 in the bottom wall 110 of the chamber 102. The ambient air and/or pure oxygen emitted through the exhaust holes 140 in the bottom surface of the chamber 102 may advantageously bubble throughout the solution in the chamber 102, thereby enhancing the interaction of oxygen with the photocatalysis reaction. One skilled in the art may select other suitable methods of enhancing the oxygenation of the photocatalysis reaction, within the scope of the present disclosure.

As shown in FIG. 1, the photoreactor system 100 may include an electrical power source. For instance, the photoreactor system 100 may be configured to be electrically coupled to a wall outlet, such as a household 110-volt outlet. In another example, the photoreactor system 100 may be configured to be solar powered. As a non-limiting example, the photoreactor system 100 may include a solar cell 142 that is configured to convert ultraviolet light into direct current energy. In a particular embodiment, the photoreactor system 100 may include a battery to store electrical energy received by the electrical power source and/or the solar cell 142. The solar cell 142 may be coupled to the lid 104 and/or the chamber 102. The solar cell 142 may be electrically coupled with the mixing blade 122 so that the mixing blade 122 may be powered by the solar cell 142.

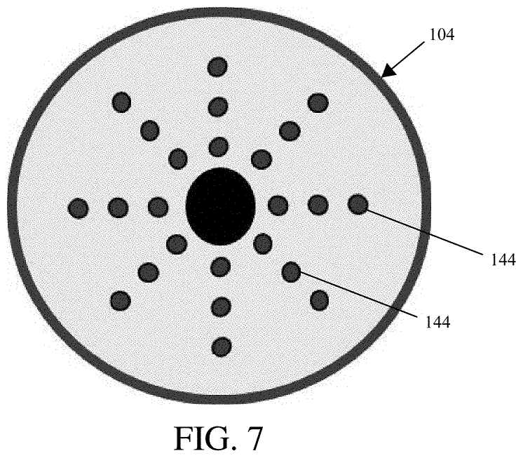

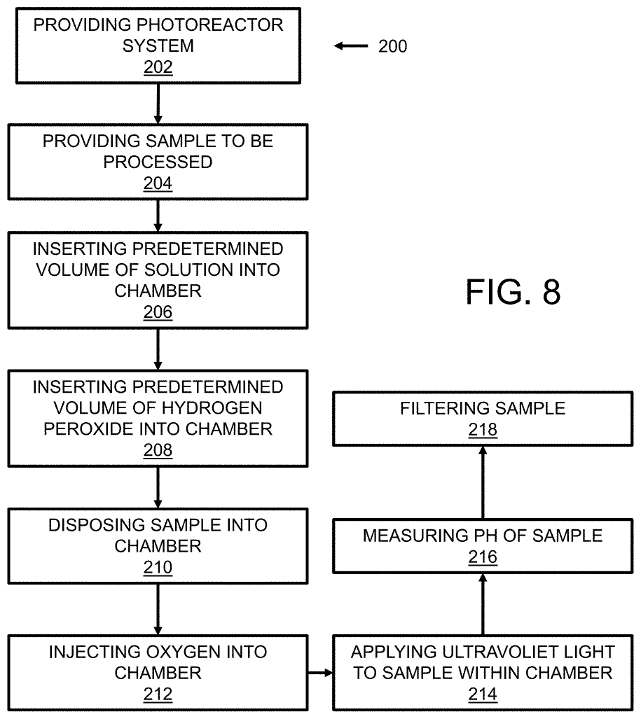

As shown in FIGS. 1 and 6-7, the photoreactor system 100 may include an ultraviolet (UV) light source and/or the photoreactor system 100 may be configured to accept UV light. For instance, the lid 104 may be a transparent lid and/or a translucent lid that is configured to permit UV light from an external source, such as the sun, to pass through the lid 104 and emit ultraviolet light into the chamber 102. Advantageously, the photoreactor system 100 may be operated more economically and sustainably where the photoreaction utilizes UV light from the sun and/or another external source. In an alternative example, the photoreactor system 100 may include an UV light source 144 coupled to an interior surface of the lid 104, an interior surface of the sidewall 108 of the chamber 102, and/or an interior surface of the bottom wall 110 of the chamber 102. In a particular embodiment, the UV light source 144 may be an UVA LED light coupled to the interior surface of the lid 104 and configured to emit the UV light into the chamber 102. In a more specific example, the UVA LED light may include a plurality of UVA LED lights. In an even more specific example, plurality of UVA LED lights may be oriented in a substantially star-shaped pattern on the lid 104. In a specific example, the UV light source 144 may include UVB and UVC LED lights, which may also be utilized to enhance the sterilization of bacterial contamination. Where the UV light source 144 is coupled to an interior surface of the lid 104, an interior surface of the sidewall 108 of the chamber 102, and/or an interior surface of the bottom wall 110 of the chamber 102, the lid 104 may be configured to reflect UV light back into the chamber 102. For instance, the interior surface of the lid 104 may be coated with a reflective coating. In a specific example, the interior surface of the lid 104 may be partially coated with the reflective coating. In a more specific example, the interior surface of the lid 104 may be entirely coated with the reflective coating. Desirably, the use of the UV light source 144 and a reflective coating on the lid 104 may enhance the efficiency of the photoreactor system 100 by preserving the UV light within the chamber 102 and minimizing the required time for the photoreactor to process the sample. In a specific example, the chamber 102 may include a height H predetermined to enhance the efficiency of the photoreactor system 100. Where the height H of the chamber 102 is reduced, the available photons have less distance to travel. In certain circumstances, up to 30% of the available photons may be reduced in the first 2.5 cm of the solution in the chamber 102. As a non-limiting example, the height H of the chamber 102 may be less than around five inches. In a more particular embodiment, the lid 104 may also be coated with barium. One skilled in the art may select any other suitable range of heights for the chamber 102, within the scope of the present disclosure.

The photoreactor system 100 may include ways to enhance the processing of the sample. Processing the sample may enabling a photocatalysis reaction to break down organic molecules such as acetaldehyde, chloroform, and ethanol. The photoreactor system 100 may also neutralize hydrochloric acid. The photoreactor system 100 may further destroy Δ-9 THC and the Duquenois-Levine chromophore. For instance, the interior surface of the sidewall 108 of the chamber 102 and/or the interior surface of the bottom wall 110 of the chamber 102 may be coated with a catalyst including TiO₂ and/or barium. The photoreactor system 100 may be configured to enhance the photocatalytic reaction by involving activating anatase TiO₂ with UV light. In certain circumstances, the coating may be coupled to the inner surface of the sidewall 108 and/or the inner surface of the bottom wall 110 with an adhesive. More particularly, the adhesive may be applied to the interior surface of the sidewall 108 of the chamber 102 and/or the interior surface of the bottom wall 110 of the chamber 102. Then, the coating having the catalyst including TiO₂ may be applied over the adhesive so that the coating having the catalyst including TiO₂ may be exposed while militating against the adhesive from impeding the reaction by covering the coating having the catalyst including TiO₂. Advantageously, the coating having the catalyst including TiO₂ may have a greater surface area exposed to the chamber 102 where the coating having the catalyst including TiO₂ is applied over the adhesive.

Various ways of using the photoreactor system 100 are provided. For instance, a method 200 may include a step 202 of providing the photoreactor system 100. The method 200 may include a step 204 of providing a sample to be processed. Next, a predetermined volume of a solution, such as water, may be inserted into the chamber 102. The method 200 may include a step 210 of disposing the sample into the chamber 102 of the photoreactor system 100. Then, oxygen may be injected into the chamber 102. Afterwards, ultraviolet light may be applied to sample within the chamber 102. A skilled artisan may select other suitable ways to use the photoreactor system 100, within the scope of the present disclosure.

In certain circumstances, the method 200 may further include various other steps. For example, the method 200 may include inserting a predetermined volume of hydrogen peroxide into the chamber 102. In another specific example, the sample may be filtered to remove solids more easily. In a particular embodiment, the method 200 may include a step 216 of measuring a pH of the sample. It should be appreciated that the steps of the method 200 may be performed in various orders.

Advantageously, the photoreactor system 100 may effectively break down the chemical by-products of THC testing kits by enhancing the surface area of the catalyst coating including TiO₂ throughout the chamber 102 and improving the agitation of the sample through the addition of the baffle 124, the mixing blade 122, and the exhaust holes in the chamber 102. Desirably, the photoreactor system 100 may be manufactured and operated more economically and efficiently.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. A photoreactor system for breaking down Tetrahydrocannabinol (THC) from a sample, comprising:
a chamber that includes a sidewall and a bottom wall;
a lid that is coupled to the sidewall of the chamber, the lid configured to be selectively disposed in at least one of an open position and a closed position;
a catalyst coating including titanium dioxide coupled to at least one of an inner surface of the sidewall and an inner surface of the bottom wall; and
an oxygen supply port coupled to the chamber.

2. The photoreactor system of claim 1, further comprising a spectrophotometric detector.

3. The photoreactor system of claim 2, wherein the spectrophotometric detector includes a view port and a device holder.

4. The photoreactor system of claim 1, further comprising a pH meter, configured to read the pH of the sample inside of the chamber.

5. The photoreactor system of claim 1, further comprising a thermometer configured to measure a temperature of the sample inside of the chamber.

6. The photoreactor system of claim 1, further comprising a mixing blade.

7. The photoreactor system of claim 7, further comprising a solar cell coupled to one of the lid and the chamber, wherein the solar cell is configured to power the mixing blade.

8. The photoreactor system of claim 1, further comprising a baffle on at least one of the inner surface of the sidewall and the inner surface of the bottom wall.

9. The photoreactor system of claim 9, wherein the baffle is at least one of substantially cylindrically shaped and substantially conically shaped.

10. The photoreactor system of claim 1, wherein the lid is one of a transparent lid and a translucent lid.

11. The photoreactor of claim 1, wherein at least a portion of an interior surface of the lid is reflective.

12. The photoreactor of claim 1, wherein an ultraviolet light is coupled to an interior surface of the lid.

13. The photoreactor system of claim 1, wherein the catalyst coating including titanium dioxide is coupled to the at least one of the inner surface of the sidewall and the inner surface of the bottom wall with an adhesive.

14. The photoreactor of claim 3, further comprising a light source configured to provide white light inside of the chamber.

15. The photoreactor of claim 14, wherein the light source provides emits light at a wavelength between 570 and 590 nm.

16. The photoreactor of claim 14, wherein the light source is adjacent to the view port.

17. The photoreactor of claim 16, wherein the light source is a first powered light and a second powered light, wherein the view port is disposed in between the first powered light and the second powered light.

18. The photoreactor of claim 16, wherein a reflective plate is disposed inside of the chamber, the reflective plate facing the light source and the view port.

19. The photoreactor of claim 1, further comprising an air pump configured to provide air to the chamber.

20. The photoreactor of claim 19, further comprising a plurality of exhaust holes fluidly connected to the pump, wherein the exhaust holes receive air pressurized by the air pump and release the air into the chamber.
